(19) United States
(12) Patent Application Publication
(10) Pub. No.: US 2026/0021258 A1
(43) Pub. Date: Jan. 22, 2026

(54) Depth Adjustable Syringe

(71) Applicants: Thomas J. Shaw, Frisco, TX (US); Retractable Technologies, Inc., Little Elm, TX (US)

(72) Inventors: Thomas J. Shaw, Frisco, TX (US); Jordan Duesman, Pilot Point, TX (US)

(21) Appl. No.: 19/271,326

(22) Filed: Jul. 16, 2025

Related U.S. Application Data
(60) Provisional application No. 63/672,500, filed on Jul. 17, 2024.

(51) Int. Cl. A61M 5/46; A61M 5/31; A61M 5/32

(52) U.S. Cl. CPC A61M 5/46; A61M 5/3137; A61M 5/3243

(57) ABSTRACT

Systems and methods are disclosed that include providing a depth adjustable syringe having a barrel with a cylindrical body portion that defines a fluid chamber and a pair of opposing substantially flat depth guides that extend laterally from the cylindrical body portion. The depth guides include longitudinally extending rails, a plurality of detents, and safety stops disposed along an inner surface. A depth adjuster includes opposing channels that slidably engage the rails of the depth guides to position the depth adjuster along the barrel based on a desired depth of an injection. The depth adjuster engages the detents in a plurality of depth injection positions and engages the safety stops in a needle safety position, in which the needle guard at least partially surrounds a tip of a needle, and the syringe is disabled for subsequent use.

### CROSS-REFERENCE TO RELATED APPLICATIONS

This application claims priority under 35 U.S.C. § 119(e) to U.S. Provisional Patent Application No. 63/672,500 filed on Jul. 17, 2024 by Thomas J. Shaw and entitled “Depth Adjustable Syringe,” the disclosure of which is hereby incorporated by reference in its entirety for all purposes.

### BACKGROUND

Medical syringes are used for administering injections or infusions of drugs, medicines, and vaccines all over the world. In an effort to control the spread of blood-borne pathogens and illnesses and the incidence of contamination by contact with either exposed needles or bodily fluids, syringes have been designed to embody various safety features, such as retractable needles and needle covers. These features are useful in protecting medical personnel from accidental “sticks” when administering injections or infusions. Additionally, different types of injections typically require a different depth of penetration into the patient, ranging from intravenous injections that are injected directly into a vein, intradermal injections that are injected into a middle layer of the skin called the dermis, subcutaneous injections that are injected into the innermost layer of the skin, and intramuscular injections that penetrate through the skin and are injected into muscular tissue, where the medicinal fluid can be absorbed quickly by the surrounding blood vessels.

These different types of injections all require a precision penetration depth to ensure the injection is given at an adequate depth in order to reach the desired tissue for the injection and also to ensure that the injection is not administered at too deep of a depth, which could result in additional pain to a patient, delayed or slow absorption of the medicinal fluid, or even rupture of a vein of the patient. For example, precision injections, such as botulinum neurotoxin injections, are often administered intradermally into the lower layers of the skin, which is much shallower than a traditional flu vaccine that is typically administered intramuscularly and penetrates into the muscle beneath the skin. Thus, the medical industry continues to demand advances in syringe technology that aid the user in administering the injection at a desired depth, thereby increasing confidence and safety for both the patient and the user.

### SUMMARY

Embodiments of a syringe disclosed herein include providing the syringe with a barrel including a cylindrical body portion that defines a fluid chamber, a nose end disposed at a forward end of the cylindrical body portion, a finger flange disposed a rearward end of the cylindrical body portion, and a pair of opposing substantially flat depth guides that extend laterally from the cylindrical body portion, wherein each of the depth guides includes a longitudinally extending rail, a plurality of detents, and a safety stop disposed along an inner surface of the depth guide; a needle received within and extending from the nose end of the barrel; a plunger assembly slidably engaging the fluid chamber defined by the cylindrical body portion of the barrel; and a depth adjuster including a main body portion, a pair of longitudinally extending channels formed in the main body portion, and a needle guard, wherein the pair of longitudinally extending channels are configured to provide sliding engagement with the longitudinally extending rails of the depth guides to slide the depth adjuster along the barrel, and wherein the depth adjuster is configured to engage a first pair of the plurality of detents in a maximum depth injection position, a second pair of the plurality of detents in at least one intermediate injection depth position, and the safety stops in a needle safety position in which the needle guard at least partially surrounds a tip of the needle.

Embodiments of a syringe disclosed herein include providing the syringe with a barrel including a cylindrical body portion that defines a fluid chamber, a nose end disposed at a forward end of the cylindrical body portion, a finger flange disposed a rearward end of the cylindrical body portion, and a pair of opposing substantially flat depth guides, wherein the depth guides include maximum depth injection position detents, intermediate depth injection position detents, and safety stops; a needle received within and extending from the nose end of the barrel; a plunger assembly slidably engaging the fluid chamber defined by the cylindrical body portion of the barrel; and a depth adjuster including a main body portion and a needle guard, wherein the depth adjuster is configured to engage the depth guides to slide the depth adjuster along the barrel to position the depth adjuster to a desired injection depth, wherein the depth adjuster is configured to engage the maximum depth injection position detents in a maximum depth injection position, the intermediate depth injection detents in an intermediate injection depth position, and the safety stops in a needle safety position.

Embodiments of a method of administering an injection with embodiments of a syringe disclosed herein include: providing a syringe including: a barrel including a cylindrical body portion that defines a fluid chamber, a nose end disposed at a forward end of the cylindrical body portion, a finger flange disposed a rearward end of the cylindrical body portion, and a pair of opposing substantially flat depth guides that extend laterally from the cylindrical body portion, wherein each of the depth guides includes a longitudinally extending rail, a plurality of detents, and a safety stop disposed along an inner surface of the depth guide; a needle received within and extending from the nose end of the barrel; a plunger assembly slidably engaging the fluid chamber defined by the cylindrical body portion of the barrel; and a depth adjuster including a main body portion, a pair of longitudinally extending channels formed in the main body portion, and a needle guard, wherein the pair of longitudinally extending channels are configured to provide sliding engagement with the longitudinally extending rails of the depth guides to slide the depth adjuster along the barrel; drawing a medicine through the needle into the fluid chamber of the barrel by activation of the plunger assembly in a first direction; sliding the depth adjuster along the barrel to a position corresponding to a desired depth for administering the injection; inserting the needle into a patient until the needle guard contacts skin of the patient; and injecting a fluid from the fluid chamber of the barrel through the needle by activation of the plunger assembly in a second opposing direction.

### BRIEF DESCRIPTION OF THE DRAWINGS

For a more complete understanding of the present disclosure and so that the manner in which the features and advantages of the embodiments can be understood in more detail, reference is now made to the following brief description, taken in connection with the accompanying drawings and detailed description.

FIG. 1 shows an oblique exploded view of a syringe according to an embodiment of the disclosure.

FIG. 2 shows an oblique assembled view of the syringe according to an embodiment of the disclosure.

FIG. 3 shows an orthogonal right-side view of the syringe configured for a maximum depth injection according to an embodiment of the disclosure.

FIG. 4 shows a cross-sectional right-side view of the syringe configured for the maximum depth injection according to an embodiment of the disclosure.

FIG. 5 shows an orthogonal right-side view of the syringe configured for an intermediate depth injection according to an embodiment of the disclosure.

FIG. 6 shows a cross-sectional right-side view of the syringe configured for the intermediate depth injection according to an embodiment of the disclosure.

FIG. 7 shows an orthogonal right-side view of the syringe configured in a safe position according to an embodiment of the disclosure.

FIG. 8 shows a cross-sectional right-side view of the syringe configured in the safe position according to an embodiment of the disclosure.

FIG. 9 shows an oblique view of the syringe configured in the safe position and having a needle cap according to an embodiment of the disclosure.

FIG. 10 shows a cross-sectional right-side view of the syringe configured in the safe position and having the needle cap according to an embodiment of the disclosure.

FIG. 11 shows a flowchart of a method of administering an injection with a syringe according to an embodiment of the disclosure.

The use of the same reference symbols in different drawings indicates similar or identical items.

### DETAILED DESCRIPTION

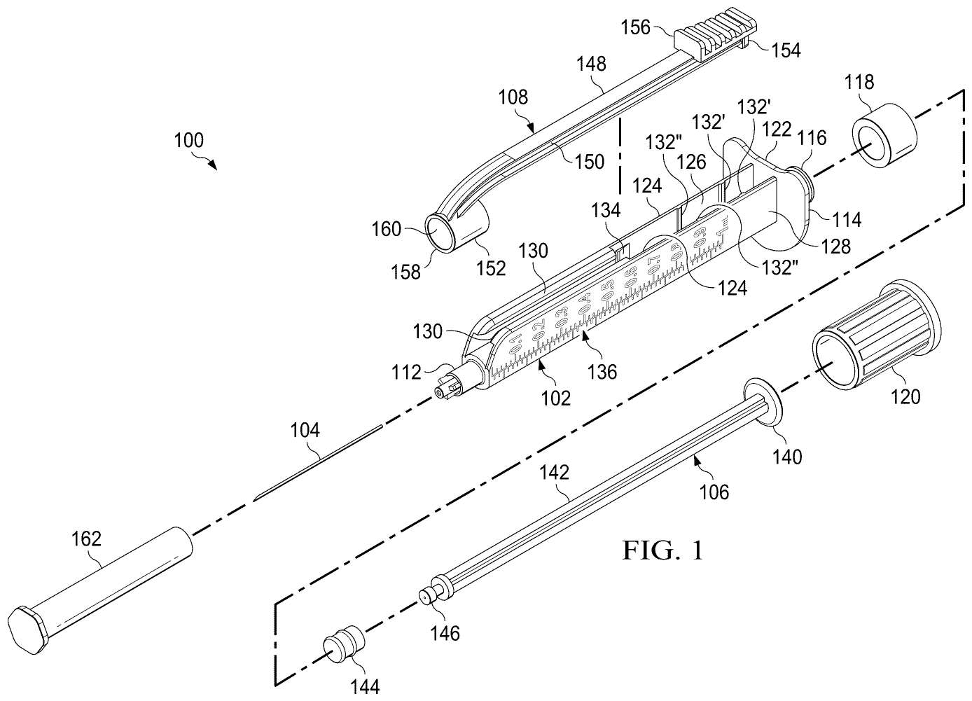

Referring to FIGS. 1 to 10, various views of a syringe 100 are shown according to an embodiment of the disclosure. The syringe 100 may generally comprise a barrel 102, a needle 104 coupled to the barrel 102, a plunger assembly 106 disposed within the barrel 102, and a depth adjuster 108 slidably engaging the barrel 102. The barrel 102 may generally comprise a cylindrical body portion 110 that defines a fluid chamber within the barrel 102. The barrel 102 may comprise a nose end 112 disposed at the forward end of the cylindrical body portion 110 for receiving and securing the needle 104 within the barrel 102. The barrel 102 may also comprise an annularly projecting finger flange 114. A cylindrical collar 116 may extend rearwardly from the finger flange 114 and provide a rearwardly facing opening that is coaxially aligned and communicates with the fluid chamber defined by the cylindrical body portion 110 of the barrel 102. The plunger assembly 106 may be inserted forwardly through the cylindrical collar 116 and into sliding engagement with the fluid chamber of the cylindrical body portion 110 of the barrel 102. An elastomeric bushing 118 may be disposed around the cylindrical collar 116 to releasably secure a plunger cap 120 in place with respect to the barrel 102. In some embodiments, the finger flange 114 may comprise a thumb notch 122 that may allow access to the depth adjuster 108.

The barrel 102 may further comprise a pair of opposing depth guides 124 that extend from the cylindrical body portion 110. The opposing depth guides 124 may be substantially flat and disposed in a parallel relationship, such that each depth guide 124 comprises an inner surface 126 and an outer surface 128. As such, the depth guides 124 may extend substantially laterally and/or tangentially from the cylindrical body portion 110 of the barrel 102. In some embodiments, the opposing depth guides 124 may extend forwardly from the finger flange 114 to the nose end 112 of the barrel 102. As such, the opposing depth guides 124, the cylindrical body portion 110, the finger flange 114, and the cylindrical collar 116 may be integrally formed in a single manufacturing operation to a form a unitarily molded, one-piece barrel 102.

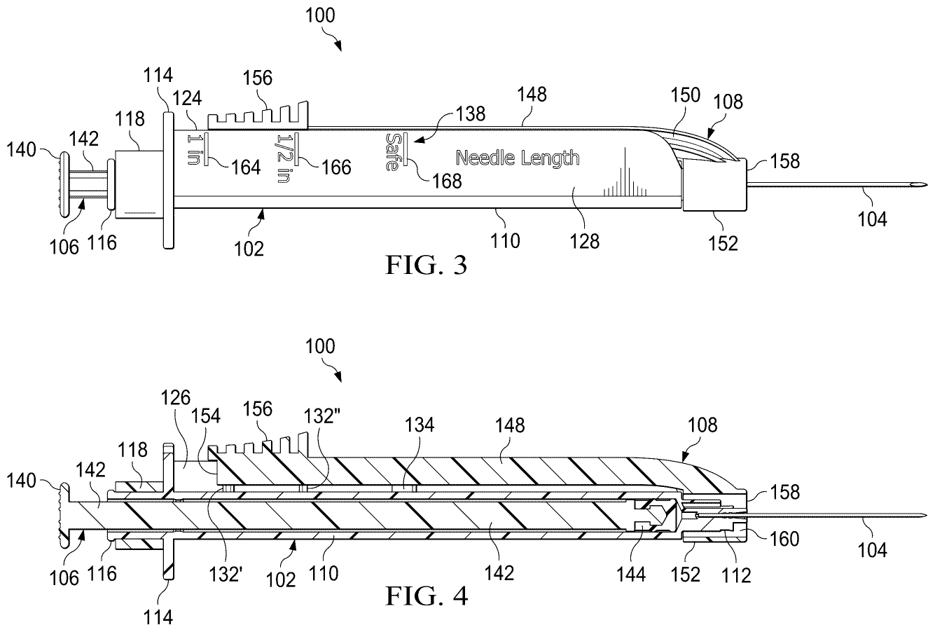

In some embodiments, each of the inner surfaces 126 of the depth guides 124 may comprise an inwardly projecting rail 130 extending longitudinally along the respective depth guide 124. In some embodiments, the rails 130 may be substantially parallel to provide a sliding interface with the depth adjuster 108. Additionally, as will be described herein with respect to some embodiments, each of the inner surfaces 126 may also comprise a plurality of detents 132 and a safety stop 134. In some embodiments, one of the depth guides 124 may comprise an outer surface 128 that is marked with an easily readable volumetric scale 136 (e.g., FIGS. 1, 2, and 9) adjacent to the cylindrical body portion 110 of the barrel 102 to facilitate reading of a liquid level inside the fluid chamber of the cylindrical body portion 110 of the barrel 102. Additionally, in some embodiments, the opposing depth guide 124 may comprise an outer surface 128 that is marked with a depth scale 138 (e.g., FIGS. 3, 5, and 7) indicating various injection depths or intervals for which the syringe 100 may be configured to provide an injection. Further, in some embodiments, the depth scale 138 may also indicate a safe position when the depth adjuster 108 has been advanced to its most forward position to disable the syringe 100 for subsequent use.

The plunger assembly 106 may generally comprise a plunger surface 140 for selectively actuating the plunger assembly 106, an elongated plunger body 142 extending therefrom, and a plunger seal 144 disposed on or about a smaller diameter projection 146 at a distal end of the elongated plunger body 142 of the plunger assembly 106. The elongated plunger body 142 may be at least partially received within the cylindrical body portion 110 of the barrel 102, such that the plunger seal 144 forms a fluid tight seal between plunger assembly 106 and the barrel 102, and more specifically, between the plunger seal 144 and the fluid chamber formed by the cylindrical body portion 110 of the barrel 102.

The depth adjuster 108 may generally be configured to slidably engage the barrel 102 to provide a plurality of depth adjustments for an injection with the syringe 100. The depth adjuster 108 may comprise a longitudinally extending main body portion 148 comprising channels 150 disposed in opposing sides of and that extend longitudinally along the main body portion 148 and are configured to provide sliding engagement with the rails 130 of the depth guides 124. A forward end of the main body portion 148 may be curved downwardly and coupled to an integrally molded, cylindrical needle guard 152. In some embodiments, a rearward end of the main body portion 148 may comprise a catch tab 154 that may extend downwardly below the channels 150 and that cooperates with the detents 132 and the safety stop 134 to provide depth adjustment and safe disable the syringe 100 after use. The rearward end of the main body portion 148 may also comprise an upwardly extending activation pad 156 used to slide the depth adjuster 108 along the barrel 102. In some embodiments, the activation pad 156 may be angled rearwardly, such that forward motion of a user's thumb through the thumb notch 122 in the finger flange 114 may catch the activation pad 156 to allow ergonomic sliding movement of the depth adjuster 108 along the barrel 102.

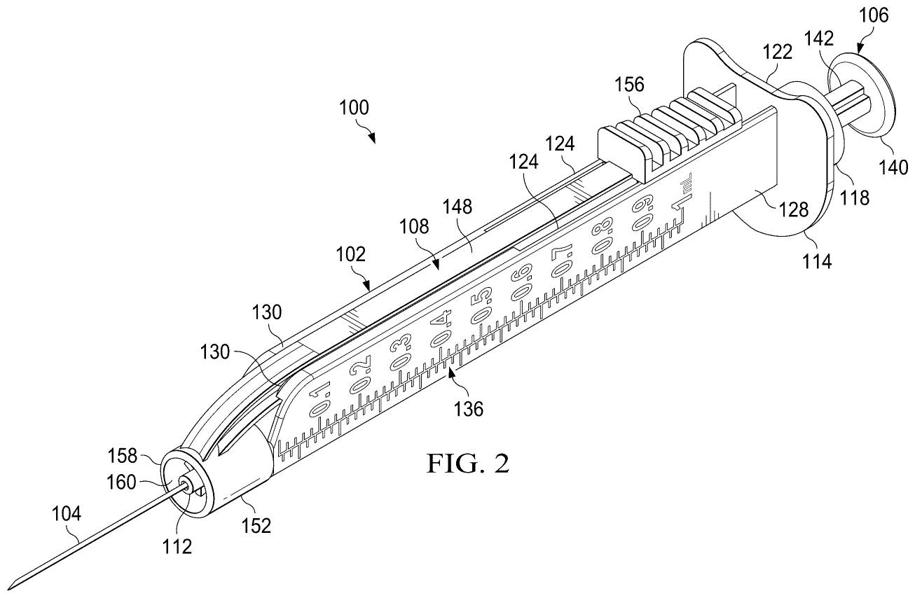

Referring to FIGS. 2 to 4, an oblique view, an orthogonal right-side view, and a cross-sectional right-side view of the syringe 100 configured for a maximum depth injection is shown according to an embodiment of the disclosure. When the syringe 100 is configured for a maximum depth injection, the depth adjuster 108 may be disposed at its most rearward position. In this position, the depth adjuster 108 and/or the catch tab 154 may be engaged with the first set of detents 132′, and a rear portion (or other portion in other embodiments) of the activation pad 156 may align with a first depth indicator 164 on the depth scale 138 indicating the depth setting (in this case, 1 inch) to the user. The first set of detents 132′ may function to retain the depth adjuster 108 in the most rearward position during the injection and/or until the depth adjuster 108 receives forwardly acting thumb pressure on the activation pad 156 sufficient to overcome a retaining strength of the first set of detents 132′ and disengage the catch tab 154 from the detents 132′. To administer the maximum depth injection, the needle 104 may be inserted into a patient until an end 158 of the needle guard 152 contacts the skin of the patient. Upon contact of the end 158 of the needle guard 152 with the patient, the desired depth of injection may be reached, and the injection may be administered by activation of the plunger assembly 106.

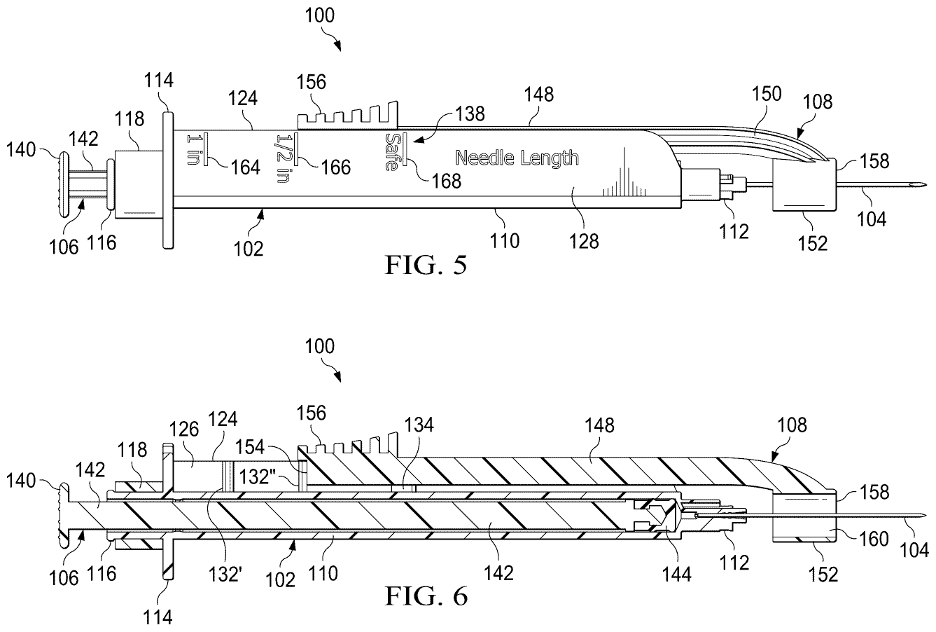

Referring to FIGS. 5 and 6, an orthogonal right-side view and a cross-sectional right-side view of the syringe configured for an intermediate depth injection is shown according to an embodiment of the disclosure. To adjust the injection depth from the maximum depth injection to the intermediate injection depth, the depth adjuster 108 may be slid forward via forwardly acting thumb pressure on the activation pad 156 sufficient to overcome a retaining strength of the first set of detents 132′ and disengage the catch tab 154 from the detents 132′. The longitudinally extending, inwardly projecting rails 130 on the inner surfaces 126 of the depth guides 124 may be configured to provide a smooth, slidable interface with the channels 150 of the depth adjuster 108 to adjust the desired depth of the injection until the depth adjuster 108 and/or the catch tab 154 engage with the second set of detents 132″, and a rear portion (or other portion in other embodiments) of the activation pad 156 aligns with an intermediate depth indicator 166 on the depth scale 138 indicating the depth setting (in this case ½ inch) to the user.

The intermediate set of detents 132″ may function to retain the depth adjuster 108 in the intermediate position during the injection and/or until the depth adjuster 108 receives forwardly acting thumb pressure on the activation pad 156 sufficient to overcome a retaining strength of the intermediate set of detents 132″ and disengage the catch tab 154 from the detents 132″. To administer the maximum depth injection, the needle 104 may be inserted into a patient until an end 158 of the needle guard 152 contacts the skin of the patient. Upon contact of the end 158 of the needle guard 152 with the patient, the desired depth of injection may be reached, and the injection may be administered by activation of the plunger assembly 106.

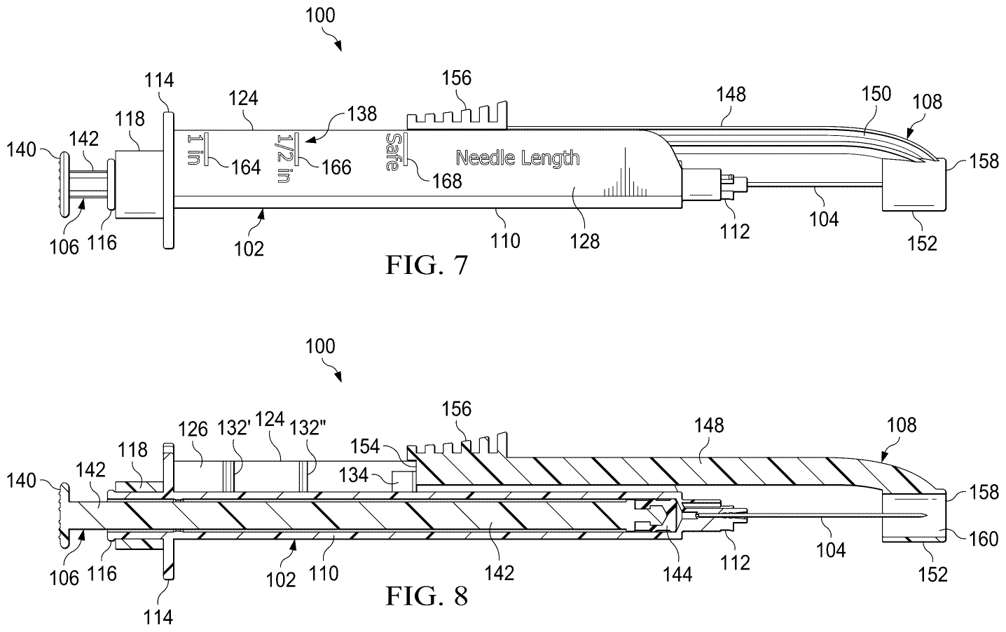

Referring to FIGS. 7 and 8, an orthogonal right-side view and a cross-sectional right-side view of the syringe configured in a safe position is shown according to an embodiment of the disclosure. Following an injection, the depth adjuster 108 may be slid forward via forwardly acting thumb pressure on the activation pad 156 sufficient to overcome a retaining strength of the intermediate set of detents 132″ and disengage the catch tab 154 from the detents 132″. The longitudinally extending, inwardly projecting rails 130 on the inner surfaces 126 of the depth guides 124 provide a smooth, slidable interface with the channels 150 of the depth adjuster 108 until the depth adjuster 108 and/or the catch tab 154 slide over an inclined portion of the safety stops 134 and engage the safety stops 134, and a rear portion (or other portion in other embodiments) of the activation pad 156 aligns with a safety indicator 168 on the depth scale 138 indicating the to the user that the syringe 100 is in the safe position and cannot be subsequently used.

In the safe position, the tip of the needle 104 may be disposed within an inner bore 160 of the depth adjuster 108, thereby circumferentially surrounding the tip of the needle 104 to prevent unintended needle sticks or inadvertent contact with the needle 104. Additionally, the safety stops 134 may engage the depth adjuster 108 and prevent subsequent rearward movement of the depth adjuster 108 to prevent subsequent use of the syringe 100 following administration of an injection. Accordingly, the depth adjuster 108 functions as a device to adjust both the depth of the injection and a safety device to disable the syringe 100 after use to prevent subsequent use of the syringe 100.

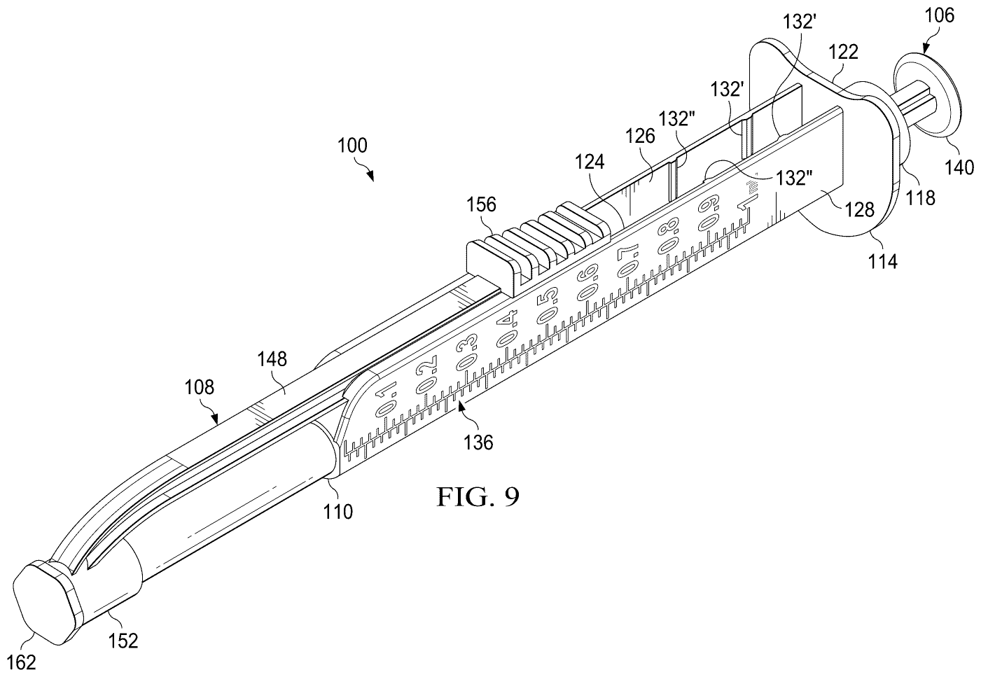

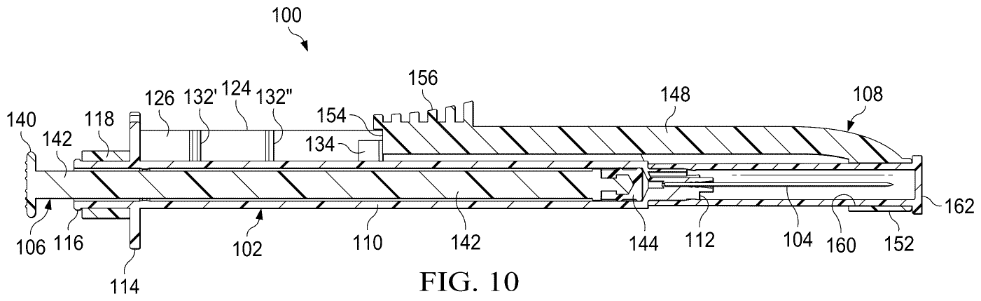

Referring to FIGS. 9 and 10, an oblique view and a cross-sectional right-side view of the syringe configured in the safe position and having the needle cap is shown according to an embodiment of the disclosure. After administration of the injection with the syringe 100, the needle cap 162 may be inserted into the inner bore 160 of the needle guard 152 of the depth adjuster 108 and over the nose end 112 of the barrel 102. The needle cap 162 may be retained by the nose end 112 of the barrel 102, thereby adding an additional safety element to the syringe 100.

Embodiments of a syringe disclosed herein are shown with a maximum depth position (1-inch depth), one intermediate position (½-inch depth), and a safe position. However, in some embodiments, the syringe 100 may comprise a plurality of intermediate positions (e.g., two, three, four, five, six, seven, eight, etc.) depending on the size and intended application for the syringe 100. For example, embodiments of a syringe 100 used for precision injections, such as botulinum neurotoxin injections that are administered intradermally into the lower layers of the skin, may comprise a maximum depth position (1-inch depth) and a plurality of intermediate positions disposed in ¼-inch or ⅛-inch increments. However, embodiments of a syringe 100 designed for administration of injections that are typically administered intramuscularly and penetrate into the muscle beneath the skin may comprise a maximum depth position (6-inch, 5-inch, 4-inch, 3-inch, 2-inch, or even 1-inch) and a plurality of intermediate positions disposed in ¼-inch, ½-inch, 1-inch, or even greater increments. Further, during advancement of the depth adjuster 108, audible clicks may indicate to a user when the position of the depth adjuster 108 is advanced to another position and/or when the depth adjuster 108 has advanced beyond a set of detents 132 and/or the safety stop 134.

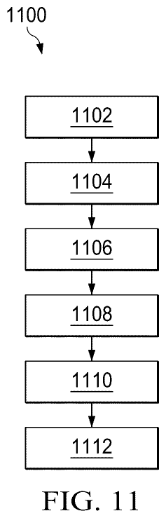

FIG. 11 shows a flowchart of a method 1100 of administering an injection with a syringe 100 according to an embodiment of the disclosure. The method 1100 may begin at block 1102 by providing a syringe 100 as disclosed herein. In some embodiments, the syringe 100 may comprise a barrel 102, a needle 104 extending from the barrel 102, a plunger assembly 106 disposed within the barrel 102, and a depth adjuster 108 slidably engaging the barrel 102. The method 1100 may continue at block 1104 by drawing a medicine through the needle 104 into the fluid chamber of the barrel 102 by activation of the plunger assembly 106 in a first direction. The method 1100 may continue at block 1106 by sliding the depth adjuster 108 along the barrel 102 to a position corresponding to a desired depth for administering the injection. In some embodiments, the desired depth for administering the injection may comprise the depth that the needle 104 penetrates below the surface of the skin of a patient receiving the injection. The method may continue at block 1108 by inserting the needle into a patient receiving the injection until the end 158 of the needle guard 152 contacts the skin of the patient. In some embodiments, the desired depth of injection is reached when the needle guard 152 contacts the skin of the patient.

The method may continue at block 1110 by injecting the medicine from the barrel 102 through the needle 104 by activation of the plunger assembly 106 in a second opposing direction. The method 1100 may continue at block 1112 by preventing subsequent use of the syringe 100. In some embodiments, preventing subsequent use of the syringe 100 may be accomplished by sliding the depth adjuster 108 along the barrel 102 to a safe position. In some embodiments, preventing subsequent use of the syringe 100 may be accomplished by sliding the depth adjuster 108 along the barrel 102 to the safe position until the depth adjuster 108 engages safety stops 134 and prevents subsequent rearward movement of the depth adjuster 108 with respect to the barrel 102. In some embodiments, preventing subsequent use of the syringe 100 may also comprise inserting a needle cap 162 through an inner bore 160 in the needle guard 152 and over the nose of the barrel 102. In some embodiments, the needle cap 162 may be inserted through the inner bore 160 in the needle guard 152 until a flange of the needle cap 162 contacts the end 158 of the needle guard 152.

In some embodiments, a depth adjustable syringe and method herein are particularly well suited for certain types of injectable medicines or medical preparations that are used for various medical or cosmetic treatments. Such medicines or medical preparations may comprise botulinum toxin type A, onabotulinumtoxinA (such as Botox®), abobotulinumtoxinA (such as Dysport®), incobotulinumtoxinA (such as Xcomin®), prabotulinumtoxinA-xvfs (such as Jeuveau®), hyaluronic acid, calcium hydroxyapatite, ply-L-lactic acid, autologous fat, collagen, polymethylmethacrylate, or any other composition suitable for cosmetic or medical use on a human subject.

### EXAMPLES

It will be appreciated that embodiments of a depth adjustable syringe 100 and/or a method 1100 of operating a depth adjustable syringe 100 may include one or more of the following Examples:

Example 1. A syringe, comprising: a barrel comprising a cylindrical body portion that defines a fluid chamber, a nose end disposed at a forward end of the cylindrical body portion, a finger flange disposed a rearward end of the cylindrical body portion, and a first depth guide, a second depth guide spaced apart from the first depth guide, wherein the first depth guide and the second depth guide are substantially flat and extend laterally from the cylindrical body portion; a needle received within and extending from the nose end of the barrel; a plunger assembly slidably engaging the fluid chamber defined by the cylindrical body portion of the barrel; and a depth adjuster comprising a main body configured to slidably engage with the first depth guide and the second depth guide, and a needle guard; a plurality of detents corresponding to a maximum depth injection position, an intermediate depth injection position, and a needle safety position in which the needle guard at least partially surrounds a tip of the needle, wherein the plurality of detents are disposed on (1) (a) the first depth guide, (b) the second depth guide, or (c) both or (2) the main body of the depth adjuster; wherein the plurality of detents are configured to selectively set a position of the needle guard relative to the needle, as the depth adjuster slidably engages with the first depth guide and the second depth guide, to a correspond to a desired injection depth or to the needle safety position.

Example 2. The syringe of Example 1, wherein the first depth guide comprises a first longitudinally extending rail, the second depth guide comprises a second longitudinally extending rail, and the main body of the depth adjuster comprises a first longitudinally extending channel configured to slidably engage with the first longitudinally extending rail and a second longitudinally extending channel configured to slidably engage with the second longitudinally extending rail.

Example 3. The syringe of Example 1 wherein the first depth guide comprises a first longitudinally extending channel, the second depth guide comprises a second longitudinally extending channel, and the main body of the depth adjuster comprises a first longitudinally extending rail configured to slidably engage with the first longitudinally extending channel and a second longitudinally extending rail configured to slidably engage with the second longitudinally extending channel.

Example 4. The syringe of any one of the preceding Examples, wherein the first depth guide and the second depth guide extend substantially tangentially from the cylindrical body portion of the barrel.

Example 5. The syringe of any one of the preceding Examples, wherein the first depth guide and the second depth guide extend forwardly from the finger flange to the nose end of the barrel.

Example 6. The syringe of any one of the preceding Examples, wherein the first depth guide and the second depth guide, the cylindrical body portion, and the finger flange are integrally formed to form a unitarily molded, one-piece barrel.

Example 7. The syringe of any one of the preceding Examples, wherein at least one of first depth guide and the second depth guide comprises an outer surface marked with readable volumetric scale.

Example 8. The syringe of any one of the preceding Examples, wherein at least one of first depth guide and the second depth guide comprises an outer surface marked with a depth scale.

Example 9. The syringe of any one of the preceding Examples, wherein the depth scale indicates the maximum depth injection position, the intermediate depth injection position, and the needle safety position.

Example 10. The syringe of any one of the preceding Examples, wherein the depth adjuster is configured to align with the maximum depth injection position, the intermediate depth injection position, and the needle safety position on the depth scale to indicate a position of the depth adjuster with respect to the barrel.

Example 11. The syringe of any one of the preceding Examples, wherein the depth adjuster comprises an activation pad that extends upwardly at an angle, and is used to slide the depth adjuster along the barrel.

Example 12. The syringe of any one of the preceding Examples, wherein the finger flange comprises a thumb notch disposed adjacently to the activation pad to allow access of a user's thumb to the depth adjuster, such that forward motion of a user's thumb through the thumb notch in the finger flange catches the activation pad to allow ergonomic sliding movement of the depth adjuster along the barrel until it reaches the desired injection depth or the needle safety position.

Example 13. The syringe of any one of the preceding Examples, wherein at least one of the plurality of detents corresponding to the needle safety position comprises a safety stop configured to prevent subsequent rearward sliding movement of the depth adjuster following administration of an injection.

Example 14. The syringe of any one of the preceding Examples, wherein the depth adjuster is configured to adjust a depth at which the needle can be inserted into a subject for an injection and to be a safety device to disable the syringe after use to prevent subsequent use of the syringe and unintended needle sticks or inadvertent contact with the needle.

Example 15. The syringe of any one of the preceding Examples, wherein there are a plurality of intermediate depth injection positions.

Example 16. The syringe of any one of the preceding Examples, wherein the plurality of intermediate depth injection positions comprises at least two, at least three, at least four, at least five, at least six, at least seven, or at least eight intermediate depth injection positions.

Example 17. The syringe of any one of the preceding Examples, wherein advancement of the depth adjuster from the maximum depth injection position to the intermediate depth injection position, advancement of the depth adjuster from the intermediate depth injection position to the needle safety position, or both produces an audible clicking noise to indicate to a user when the position of the depth adjuster is advanced.

Example 18. A syringe, comprising: a barrel comprising a cylindrical body portion that defines a fluid chamber, a nose end disposed at a forward end of the cylindrical body portion, a finger flange disposed a rearward end of the cylindrical body portion, and a pair of opposing substantially flat depth guides, wherein the pair of opposing substantially flat depth guides comprise maximum depth injection position detents, intermediate depth injection position detents, and safety stops; a needle received within and extending from the nose end of the barrel; a plunger assembly slidably engaging the fluid chamber defined by the cylindrical body portion of the barrel; and a depth adjuster comprising a main body portion and a needle guard, wherein the depth adjuster is configured to engage the pair of opposing substantially flat depth guides to slide the depth adjuster along the barrel to position the depth adjuster to a desired injection depth, wherein the depth adjuster is configured to engage the maximum depth injection position detents in a maximum depth injection position, the intermediate depth injection position detents in an intermediate depth injection position, and the safety stops in a needle safety position.

Example 19. The syringe of Example 18, wherein at least one of the pair of opposing substantially flat depth guides comprises an outer surface marked with readable volumetric scale, and wherein at least one of the pair of opposing substantially flat depth guides comprises an outer surface marked with a depth scale and optionally or preferably wherein the depth scale indicates the maximum depth injection position, the intermediate depth injection position, and the needle safety position.

Example 20. The syringe of any one of Examples 18-19, wherein the depth guides extend laterally from the cylindrical body portion of the barrel.

Example 21. The syringe of any of any one of Examples 18-20, wherein the depth guides extend forwardly from the finger flange to the nose end of the barrel.

Example 22. The syringe of any one of Examples 18-21, wherein the depth guides, the cylindrical body portion, and the finger flange are integrally formed to form a unitarily molded, one-piece barrel.

Example 23. The syringe of any one of Examples 18-22, wherein at least one of the depth guides comprises an outer surface marked with readable volumetric scale.

Example 24. The syringe of any one of Examples 18-23, wherein at least one of the depth guides comprises an outer surface marked with a depth scale.

Example 25. The syringe of any one of Examples 18-24, wherein the depth scale indicates the maximum depth injection position, the intermediate depth injection position, and the needle safety position.

Example 26. The syringe of any one of Examples 18-25, wherein the depth adjuster is configured to align with the maximum depth injection position, the at least one intermediate depth injection position, and the needle safety position on the depth scale to indicate a position of the depth adjuster with respect to the barrel.

Example 27. The syringe of any one of Examples 18-26, wherein the plunger assembly comprises a plunger surface for selectively actuating the plunger assembly, an elongated plunger body extending from the plunger surface, and a plunger seal disposed on a distal end of the elongated plunger body.

Example 28. The syringe of any one of Examples 18-27, wherein the plunger seal slidably engages the fluid chamber defined by the cylindrical body portion of the barrel to form a fluid tight seal between the fluid chamber and the plunger assembly.

Example 29. The syringe of any one of Examples 18-28, wherein the depth adjuster comprises an upwardly extending activation pad used to slide the depth adjuster along the barrel.

Example 30. The syringe of any one of Examples 18-29, wherein the activation pad is angled rearwardly.

Example 31. The syringe of any one of Examples 18-30, wherein the finger flange comprises a thumb notch disposed adjacently to the activation pad to allow access of a user's thumb to the depth adjuster, such that forward motion of a user's thumb through the thumb notch in the finger flange catches the activation pad to allow ergonomic sliding movement of the depth adjuster along the barrel.

Example 32. The syringe of any one of Examples 18-31, wherein the depth adjuster is configured to engage the safety stops in the needle safety position to prevent subsequent rearward movement of the depth adjuster following administration of an injection.

Example 33. The syringe of any one of Examples 18-32, wherein the depth adjuster functions to adjust a depth of an injection and as a safety device to disable the syringe after use to prevent subsequent use of the syringe and unintended needle sticks or inadvertent contact with the needle.

Example 34. The syringe of any one of Examples 18-33, wherein the syringe comprises a plurality of intermediate injection depth positions.

Example 35. The syringe of any one of Examples 18-34, wherein the syringe comprises at least two, at least three, at least four, at least 5, at least six, at least seven, or at least eight intermediate injection depth positions.

Example 36. The syringe of any one of Examples 18-35, wherein advancement of the depth adjuster from the maximum injection depth position to the at least one intermediate injection depth position, advancement of the depth adjuster from the at least one intermediate injection depth position to the needle safety position, or both produces an audible clicking noise to indicate to a user when the position of the depth adjuster is advanced.

Example 37. A method of administering an injection, comprising: providing a syringe comprising: a barrel comprising a cylindrical body portion that defines a fluid chamber, a nose end disposed at a forward end of the cylindrical body portion, a finger flange disposed a rearward end of the cylindrical body portion, and a pair of opposing substantially flat depth guides that extend laterally from the cylindrical body portion, wherein each of the depth guides comprises a longitudinally extending rail, a plurality of detents, and a safety stop disposed along an inner surface of the depth guide; a needle received within and extending from the nose end of the barrel; a plunger assembly slidably engaging the fluid chamber defined by the cylindrical body portion of the barrel; and a depth adjuster comprising a main body portion, a pair of longitudinally extending channels formed in the main body portion, and a needle guard, wherein the pair of longitudinally extending channels are configured to provide sliding engagement with the longitudinally extending rails of the depth guides to slide the depth adjuster along the barrel; drawing a medicine through the needle into the fluid chamber of the barrel by activation of the plunger assembly in a first direction; sliding the depth adjuster along the barrel to a position corresponding to a desired depth for administering the injection; inserting the needle into a patient until the needle guard contacts skin of the patient; and injecting a fluid from the fluid chamber of the barrel through the needle by activation of the plunger assembly in a second opposing direction.

Example 38. The method of any of the preceding method Examples, further comprising: sliding the depth adjuster along the barrel until the depth adjuster engages a plurality of safety stops in a needle safety position, wherein subsequent rearward movement of the depth adjuster is prevented by the engagement of the depth adjuster with the plurality of safety stops.

Example 39. The method of any of the preceding method Examples, wherein the needle guard at least partially surrounds a tip of the needle when the depth adjuster is in the needle safety position to prevent inadvertent contact with the needle.

Example 40. The method of any of the preceding method Examples, further comprising: inserting a needle cap through an inner bore of the needle guard and over the needle and the nose of the barrel to prevent inadvertent contact with the needle.

Example 41. The method of any of the preceding method Examples wherein the syringe is according to any one of Examples 1-36.

Example 42. A method of administering an injection at a selected depth using a syringe according to any one of Examples 1-36, the method comprising: drawing a medicine or medical preparation through the needle into the fluid chamber of the barrel by activation of the plunger assembly in a first direction; sliding the depth adjuster along the barrel to a position corresponding to a desired depth for administering the injection; inserting the needle into a patient until the needle guard contacts skin of the patient; and injecting a fluid from the fluid chamber of the barrel through the needle into the patient by activation of the plunger assembly in a second opposing direction.

Example 42. The method of Example 41, further comprising sliding the dept adjuster to the needle safety position when the injecting the fluid is complete.

Example 42. The method of any one of Examples 36-42 wherein the fluid comprises botulinum toxin type A, onabotulinumtoxinA (such as Botox®), abobotulinumtoxinA (such as Dysport®), incobotulinumtoxinA (such as Xcomin®), prabotulinumtoxinA-xvfs (such as Jeuveau®), hyaluronic acid, calcium hydroxyapatite, ply-L-lactic acid, autologous fat, collagen, polymethylmethacrylate, or any other composition suitable for cosmetic or medical use on a human subject.

In the foregoing specification, the concepts have been described with reference to specific embodiments. However, those of ordinary skill in the art appreciate that various modifications and changes can be made without departing from the scope of the invention as set forth in the claims below. Other examples that occur to those skilled in the art are intended to be within the scope of the claims if they have structural elements that do not differ from the literal language of the claims, or if they include equivalent structural elements with insubstantial differences from the literal languages of the claims. Accordingly, the specification and figures are to be regarded in an illustrative rather than in a restrictive sense, and all such modifications are intended to be included within the scope of the invention.

Benefits, other advantages, and solutions to problems have been described above with respect to one or more specific embodiments. After reading the specification, those of ordinary skill in the art will appreciate that certain features that are, for clarity, described herein in the context of separate embodiments, may also be provided in combination in a single embodiment. Conversely, various features that are, for brevity, described in the context of a single embodiment, may also be provided separately or in any subcombination. Any structural elements or arrangement of structural elements relative to each other, or method steps, or medicine or medical preparation, of an embodiment, or a preferred embodiment, herein may be used with any other structural elements, features, components, medicines, medical preparations, or steps of other embodiments even if not specifically described with respect to that embodiment, unless such combination is explicitly excluded herein. Any structural element or arrangement of structural elements relative to each other, or method steps, or medicine or medical preparation of an embodiment described as excluded with any particular embodiment, or preferred embodiment, herein may similarly be excluded with any other embodiment herein even if not specifically described with such embodiment. Further, references to values stated in ranges include each and every value within that range and include sub-ranges that overlap from one preferred range to another preferred range.

What is claimed is:

1. A syringe, comprising:
a barrel comprising a cylindrical body portion that defines a fluid chamber, a nose end disposed at a forward end of the cylindrical body portion, a finger flange disposed a rearward end of the cylindrical body portion, and a first depth guide, a second depth guide spaced apart from the first depth guide, wherein the first depth guide and the second depth guide are substantially flat and extend laterally from the cylindrical body portion;
a needle received within and extending from the nose end of the barrel;
a plunger assembly slidably engaging the fluid chamber defined by the cylindrical body portion of the barrel; and
a depth adjuster comprising a main body configured to slidably engage with the first depth guide and the second depth guide, and a needle guard;
a plurality of detents corresponding to a maximum depth injection position, an intermediate depth injection position, and a needle safety position in which the needle guard at least partially surrounds a tip of the needle, wherein the plurality of detents are disposed on (1) (a) the first depth guide, (b) the second depth guide, or (c) both or (2) the main body of the depth adjuster;
wherein the plurality of detents are configured to selectively set a position of the needle guard relative to the needle, as the depth adjuster slidably engages with the first depth guide and the second depth guide, to a correspond to a desired injection depth or to the needle safety position.

2. The syringe of claim 1, wherein the first depth guide comprises a first longitudinally extending rail, the second depth guide comprises a second longitudinally extending rail, and the main body of the depth adjuster comprises a first longitudinally extending channel configured to slidably engage with the first longitudinally extending rail and a second longitudinally extending channel configured to slidably engage with the second longitudinally extending rail.

3. The syringe of claim 1 wherein the first depth guide comprises a first longitudinally extending channel, the second depth guide comprises a second longitudinally extending channel, and the main body of the depth adjuster comprises a first longitudinally extending rail configured to slidably engage with the first longitudinally extending channel and a second longitudinally extending rail configured to slidably engage with the second longitudinally extending channel.

4. The syringe of claim 1, wherein the first depth guide and the second depth guide extend substantially tangentially from the cylindrical body portion of the barrel.

5. The syringe of claim 4, wherein the first depth guide and the second depth guide extend forwardly from the finger flange to the nose end of the barrel.

6. The syringe of claim 5, wherein the first depth guide and the second depth guide, the cylindrical body portion, and the finger flange are integrally formed to form a unitarily molded, one-piece barrel.

7. The syringe of claim 1, wherein at least one of first depth guide and the second depth guide comprises an outer surface marked with readable volumetric scale.

8. The syringe of claim 7, wherein at least one of first depth guide and the second depth guide comprises an outer surface marked with a depth scale.

9. The syringe of claim 8, wherein the depth scale indicates the maximum depth injection position, the intermediate depth injection position, and the needle safety position.

10. The syringe of claim 9, wherein the depth adjuster is configured to align with the maximum depth injection position, the intermediate depth injection position, and the needle safety position on the depth scale to indicate a position of the depth adjuster with respect to the barrel.

11. The syringe of claim 1, wherein the depth adjuster comprises an activation pad that extends upwardly at an angle, and is used to slide the depth adjuster along the barrel.

12. The syringe of claim 11, wherein the finger flange comprises a thumb notch disposed adjacently to the activation pad to allow access of a user's thumb to the depth adjuster, such that forward motion of a user's thumb through the thumb notch in the finger flange catches the activation pad to allow ergonomic sliding movement of the depth adjuster along the barrel until it reaches the desired injection depth or the needle safety position.

13. The syringe of claim 1, wherein at least one of the plurality of detents corresponding to the needle safety position comprises a safety stop configured to prevent subsequent rearward sliding movement of the depth adjuster following administration of an injection.

14. The syringe of claim 13, wherein the depth adjuster is configured to adjust a depth at which the needle can be inserted into a subject for an injection and to be a safety device to disable the syringe after use to prevent subsequent use of the syringe and unintended needle sticks or inadvertent contact with the needle.

15. The syringe of claim 1, wherein there are a plurality of intermediate depth injection positions.

16. The syringe of claim 15, wherein the plurality of intermediate depth injection positions comprises at least two, at least three, at least four, at least five, at least six, at least seven, or at least eight intermediate depth injection positions.

17. The syringe of claim 1, wherein advancement of the depth adjuster from the maximum depth injection position to the intermediate depth injection position, advancement of the depth adjuster from the intermediate depth injection position to the needle safety position, or both produces an audible clicking noise to indicate to a user when the position of the depth adjuster is advanced.

18. A syringe, comprising:
a barrel comprising a cylindrical body portion that defines a fluid chamber, a nose end disposed at a forward end of the cylindrical body portion, a finger flange disposed a rearward end of the cylindrical body portion, and a pair of opposing substantially flat depth guides, wherein the pair of opposing substantially flat depth guides comprise maximum depth injection position detents, intermediate depth injection position detents, and safety stops;
a needle received within and extending from the nose end of the barrel;
a plunger assembly slidably engaging the fluid chamber defined by the cylindrical body portion of the barrel; and
a depth adjuster comprising a main body portion and a needle guard, wherein the depth adjuster is configured to engage the pair of opposing substantially flat depth guides to slide the depth adjuster along the barrel to position the depth adjuster to a desired injection depth, wherein the depth adjuster is configured to engage the maximum depth injection position detents in a maximum depth injection position, the intermediate depth injection position detents in an intermediate depth injection position, and the safety stops in a needle safety position.

19. The syringe of claim 18, wherein at least one of the pair of opposing substantially flat depth guides comprises an outer surface marked with readable volumetric scale, and wherein at least one of the pair of opposing substantially flat depth guides comprises an outer surface marked with a depth scale.

20. The syringe of claim 19, wherein the depth scale indicates the maximum depth injection position, the intermediate depth injection position, and the needle safety position.
